(19) United States
(12) Patent Application Publication
(10) Pub. No.: US 2011/0065164 A1
(43) Pub. Date: Mar. 17, 2011

(54) DOUBLE-LABELLING AGENTS BASED ON VINYL SULPHONE

(75) Inventors: Francisco Santoyo Gonzalez, Granada (ES); Fernando Hernández Mateo, Granada (ES); Francisco Javier López Jaramillo, Granada (ES); Julia Morales Sanfrutos, Granada (ES); Rafael Salto Gonzáalez, Granada (ES); Dolores Girón González, Granada (ES)

(73) Assignee: Universidad De Granada, Granada (ES)

(21) Appl. No.: 12/920,042

(22) PCT Filed: Feb. 19, 2009
(86) PCT No.: PCT/ES09/70035

§ 371 Date: Nov. 30, 2010

(30) Foreign Application Priority Data
Feb. 27, 2008 (ES) P200800565

(51) Int. Cl. C07K 1/13; C07D 495/04; C12N 9/24; C12N 9/08

(52) U.S. Cl. 435/192; 548/256; 548/255; 530/363; 435/200

(57) ABSTRACT

The invention relates to labelling agents containing a compound with two labelled molecules and a vinyl sulphone group. The invention also relates to the compounds, the method for obtaining these and the uses thereof in the marking of biomolecules and, more specifically, proteins.

The present invention refers to a compound of the general formula (I) comprising two labelled molecules and a vinyl sulphone group, whose function is to make the covalent binding with the molecules susceptible to be labelled. The present invention also refers to the procedures for obtaining them and their uses. More particularly, it refers to the use of these compounds containing simultaneously biotin and fluorophores for the labelling of biomolecules and their biotechnological applications.

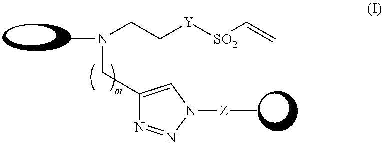

### PRIOR STATE OF THE ART

The labelling of biomolecules is a basic tool in the field of genomics and proteomics for the detection, purification and study of interactions between biomolecules.

From the range of biomolecule labellings which are plausible, there stand out by their special importance the labelling with fluorophores and biotin due to their biotechnological applications and their commercial impact.

Fluorescent labelling is a key element for the detection and analysis of biomolecules (Patton, W. F. Electrophoresis (2000), vol. 21, pp. 1123-1144) and it is the engine of a multi-million euro industry. The advantages of fluorescent labelling vis-á-vis conventional methods such as Coomassie blue, silver, colloidal gold or radioactivity are the following:
Rapid and high sensitivity detection: each fluorescent label can originate 10⁷-10⁸ photons per second.
Versatility: Different labellings originate different “colours”, being possible to make a “polychromatic” labelling such as that used, for example, in DNA sequentiation (Smith, L., et al., Nature (1986), vol. 321, pp. 674-679).
Inertia: Fluorophore size and properties rarely intervene with the marked molecule.
Localization of the signal in the labelling point, unlike enzymatic labelling.

However, its potential goes beyond passive detection since techniques such as fluorescence polarization and FRET (Fluorescence Resonance Energy Transfer, also called Förster Resonance Energy Transfer) enable to evaluate conformational changes, interactions between proteins and between protein and ligand. The measurement of the polarization provides information on orientations and mobility which enables to study receptor-ligand interactions (Jameson, D. M., Seifried, S. E., Methods (1999), vol. 19, pp. 222-233), and FRET is an interaction between fluorophores in which the excitation passes from an excited fluorophore (donor) to another which is excited (acceptor) without the emission of a photon. This interaction is produced when the donor emission wavelength is very close to that of the acceptor excitation and is very dependent on the distance between the donor and the acceptor, so it was used as rule (Remedios, C. G., Moens, P. D., J. Struct. Biol. (1995), vol. 115, pp. 175-185) to analyse conformational changes and interaction between biomolecules.

Nowadays, there exist a great amount and variety of fluorophores. Among the ones used for biomolecule labelling, we can mention dansyl, fluorescein and rhodamine B, whose functional characteristics and some of their applications are summarized in the table below:

| | Λ | Λ | |
|---|---|---|---|
| Fluorophores | absorption | emission | Some applications |
| Dansyl | 335 nm | 518 nm | Labelling for detection in general |
| | | | Quantum yield depending on the medium: |
| | | | receptor-ligand interaction analysis FRET with |
| | | | Tryptophan (donor) and with fluorescein |
| | | | (acceptor) (Gettins, P. G. W., Olson, S. T. |
| | | | Methods (2004), vol. 32, pp. 110-119) |
| Fluorescein | 494 nm | 518 nm | Labelling for detection in general |
| | | | Application in fluorescence polarization FRET |
| | | | with Rhodamine (acceptor) (Ghosh, S. S., et |
| | | | al., Nucleic Acids Res. (1994), vol. 22, pp. |
| | | | 3155-3159) |
| | | | Homo-FRET (Hamman, B. D., et al., |
| | | | Biochemistry (1996), vol. 35, pp. 16680-16686) |
| Rhodamine B | 543 nm | 565 nm | Labelling for detection in general |
| | | | Application in fluorescence polarization |
| | | | FRET with fluorescein or dansyl (donors) |
| | | | (Yegneswaran, S., et al., J. Mol. Biol. (2003), |
| | | | vol. 278, pp. 14614-14621) |

On the other hand, labelling with biotin is also very important in biotechnology (Wilchek, M., Bayer, E. A., Anal. Biochem. (1988), vol. 171, pp. 1-32). Biotin is a molecule which acts as prosthetic group of certain carboxylases related to the metabolism of carbon dioxide. However, its biotechnological interest lies in the high specificity and affinity which avidin, streptavidin and other related proteins have for this biomolecule (dissociation constant around 10⁻¹⁵ M⁻¹), causing the interaction to have the strength of a covalent bond without being one. Thus, the biotinylation transforms molecules which are hard to detect in probes which can be detected or captured with marked or immobilized avidin/streptavidin. This principle is common to find antigens in tissues, cells and to detect biomolecules in immunoassays and in DNA hybridization tests. However, for certain applications, such as for example purification through affinity chromatography, it is necessary that the biotin-avidin interaction be reversible, for which adivin can be modified (by nitrosylation of tyrosines of the active centre (Morag, E., et al., Biochem. J., (1996), vol. 316: pp. 193-199)) or biotin derivatives can be used (desthiobiotin and iminobiotin). There exist biotins fluorescently marked to quantify active sites of avidin (Gruber, H. J, et al., Biochim. Biophys. Acta (1998), vol. 1381, pp. 203-212) and biotin labelled with DNP (DNP-X-biocytin-X; U.S. Pat. No. 5,180,828A) (dinitrophenol), versatile labelling which besides acting as chromophore is recognized by antibodies anti-DNP, allowing the correlation between fluorescence and electronic microscopy studies. There also exists in the market Horseradish peroxidase (HRP) labelled with biotin.

A fundamental aspect vis-á-vis the use of any labelling is the binding to the biomolecule and the stability of said binding. From a chemical point of view there exist four groups present in the biomolecules susceptible of acting as targets for the anchorage of the labelling reagents conveniently derivatized through the formation of a covalent bond, such as amines, thiols, alcohols and carboxylic acids, which are detailed below:

Amines: They are the most common target of reagents of covalent modification and the main one in proteins. In most of these biomolecules the end amino is free and almost all have lysine, residue in the side chain of which there is a ε-amino group easily modifiable since it is mostly found in the surface of proteins. These groups react with acylating reagents and the reactivity depends on the acylating reagent, the type of amine, basicity and pH of the reaction. Aliphatic amines, such as that of the side chain of lysine, are moderately basic and react with most acylating reagents to pH higher than 8.

There are three derivatizations of labelling reagents which react with amines of biomolecules:
Succinimidyl esters: They react with amines to originate amides. It is the most frequent derivatization given the stability of the amide bond which is generated. They react well with aliphatic amines and have low reactivity with aromatic amines, alcohols, phenols (tyrosine) and imidazoles (histidine). In presence of thiols (cysteine) they can form thioesters but in proteins the acyl group can be transferred to a neighbour amine. One of the main inconveniences of succinimidyl esters is their solubility, which in some cases can be very low. Therefore, in the market there exist carboxylic acid derivatives which can be converted into succinimidyl esters (Staros, J. V., et al., Anal. Biochem. (1986), vol. 156, pp. 220-222) or STP esters (Gee, K. R., et al., Tetrahedron Lett. (1999), vol. 40, pp. 1471-1474), which are more polar, and therefore more water-soluble, although less reactive with amines with little exposure.
Isothiocyanates: They react with amines to form thioureas, which are reasonably stable in most cases.
Sulfonic acid chlorides: They react with amines and produce sulphonamides. They are very reactive and unstable in aqueous means, especially to alkaline pH necessary for them to react with aliphatic amines, so work is done at low temperature. Once conjugated, the bond is extremely stable and resistant. They also react with phenols (tyrosine), aliphatic alcohols (polysaccharides), thiols (cysteine) and imidazoles (histidine) although those conjugated with thiols and imidazoles are unstable and those conjugated with aliphatic alcohols can undergo nucleophilic displacements.
Other functionalizations can be: aldehydes and arylating agents. Aldehydes which react with amines to form Schiff bases. There have been prepared o-phthalaldehyde (OPA), naphthalene dicarboxaldehyde (NDA) and 3-acryl quinoline carboxaldehyde (OTTO-TAG) and there have been used for quantification of amines in solution (Liu, J., Hsieh, et al., Anal. Chem. (1991), vol. 163, pp. 408-412). And arylating agents such as 4-nitro-2,1,3-benzoxadiazol (NBD) chloride or fluoride (Watanable, Y., lmai, K., J. Chromatogr. (1982), vol. 239, pp. 723-732).
Thiols: They are more selective targets than the amine group, as they are less frequent in biomolecules and to be reagents they have to be free (not form a disulphide bridge). The sulfhydryl group can be introduced in the macromolecule to mark through chemical modification, reduction of disulphide bridges or intein path (Tan, L. P., Yao, S. Q., Protein and Pept. Lett. (2005), vol. 12, pp. 769-751) (in the case of proteins), or through directed mutagenesis to introduce cysteine.

Thiol groups react to physiological pH (pH 6, 5-8) with alkylating reagents (such as iodoacetamides and maleimides) or arylating reagents (such as 7-chloro or 7-fluoro-4-nitro-2,1,3-benzoxadiazol (NBD)), to originate stable thioethers. They also react with many of the acylating reagents of amines, including isothiocyanates and succinimidyl esters. Symmetric disulphides such as didansyl-L-cysteine or 5,5′-dithiobis-(2-nitrobenzoic) acid (DTNB) (Daly, T J., et al., Biochemistry (1986), vol. 25, pp. 5468-5474) also react with the thiols to give bindings of the non-symmetric disulphide type.

Alcohols: The hydroxyl function is present in the side chains of tyrosine, serine and threonine, in sterols and carbohydrates, but its reactivity in aqueous solutions is extremely low, especially in proteins due to the presence of more active nucleophiles such as amines and thiols. A function which reacts specifically with neighbour diols is boronic acid and forms cyclical complexes (Gallop, P. M., et al., Science (1982), vol. 217, pp. 166-169). However, a standard procedure to increase reactivity, especially in the case of carbohydrates, is oxidation with periodate to give origin to the aldehyde function. The main functionalizations of labelling reagents which react with the aldehyde function of biomolecules are: amine, hydrazides, semicarbazide, carbohydrazide and O-alkylhydroxylamines.

Carboxylic acids: They are abundant in macromolecules but little reactive, so their derivatization is usual so that amines are inserted which react with the functionalizations described above.

Nowadays, it is possible to commercially acquire an entire range of labelling products with conveniently derivatized fluorescence and biotin. The most frequent strategy to functionalize labelling reagents is the derivatization as succinimidyl esters to react with the amine functions of the biomolecule.

On the other hand, and from a chemical perspective, α,β-unsaturated sulphones (vinyl sulphones) are known as synthetic intermediaries greatly useful mainly because of their capacity to participate in 1,4-addition reactions (Michael acceptors). Additionally, vinyl sulphones are easy to prepare, through a wide variety of synthetic processes, and to manipulate (Simphinks, N. S., Tetrahedron (1990), vol. 282, pp. 6951-6984). These characteristics have recently been found useful in the design of drugs and in medicinal chemistry when their capacity to powerfully and reversibly inhibit a variety of enzymatic processes, mainly those involving cysteine proteases to which they are joined through addition reactions with the thiol group present in the cysteine residue of the active site of these enzymes, was proved (Meadows, D. C., et al., Med. Res. Rev. (2006), vol. 26(6), pp. 793-814).

However, from a biotechnological viewpoint, their potential goes beyond that. The reactivity of vinyl sulphones with biomolecules has been harnessed for the introduction of polyethylene glycol through reaction with thiols (Morpurgo, M., et al. Bioconiug. Chem. (1996) vol. 7, pp. 363-368), for the formation of hydrogels through peptide crosslinking with polyethylene glycol functionalized with vinyl sulphone (Rizzi, S. C, et al., Biomacromolecules (2006), vol. 7, pp. 3019-3029) and for the introduction of derivatized glucose molecules with vinyl sulphone through reaction with the amines of the proteins (Lopez-Jaramillo, et al., Acta Cryst. (2005) vol. F61, pp. 435-438).

As markers, there have been described different coloured compounds containing vinyl sulphone groups. In this sense, U.S. Pat. No. 4,473,693 describes colouring agents, for intracellular marking, based on Lucifer yellow and containing a vinyl sulphone group. In patent EP0187076 there are described fluorescent compounds containing a vinyl sulphone group, these compounds are useful for immunologic studies.

### EXPLANATION OF THE INVENTION

In the present invention it is provided a new compound of the general formula (I) comprising two different labelled molecules, and a vinyl sulphone group, which enables to perform the labelling of biomolecules in a highly efficient and simple manner. These compounds constitute an alternative to derivatizations used in proteomics and genomics to introduce a labelling reagent in biomolecules.

Therefore, a first aspect of the present invention refers to compounds of the general formula (I) (hereinafter compounds of the invention):

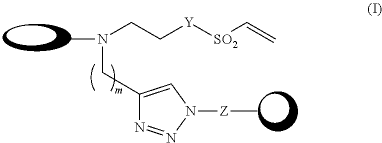

wherein:
Y is the —SO₂R— group or does not exist; where:
R is a radical, substituted or non-substituted, selected from the group comprising an alkyl (C₁-C₁₀), a dialkyl aryl ((C₁-C₁₀)Ar(C₁-C₁₀)) or a group (CH₂—CH₂O)ₙCH₂—CH₂;
where n takes values from 2 to 20;
Z is a radical, substituted or non-substituted, selected from the group comprising an alkyl (C₁-C₁₀), a dialkyl aryl ((C₁-C₁₀)Ar(C₁-C₁₀)) or a group (CH₂—CH₂O)ₙCH₂—CH₂; where n takes values from 2 to 20, n preferably takes values from 2 to 10, more preferably n is 2, 3, 4 or 5, and even more preferably n is 2.

In a preferred embodiment, Z is an alkyl group (C₁-C₅); and more preferably it is a methyl (—CH₂—) or an alkyl (—CH₂—CH₂—)

m takes values from 1 to 20 and represents an aliphatic chain (lineal or branched, substituted or non-substituted). Preferably, m takes values from 1 to 10, more preferably from 1 to 5 and even more preferably m is 1.

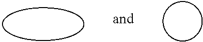

each figure independently represents a label molecule. Each molecule is of a different nature.

When Y is a —SO₂R group, the compounds of the invention have the general formula (IV):

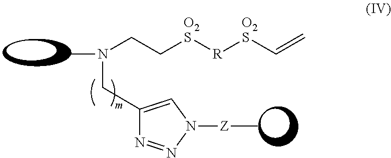

In a preferred embodiment, R is a (—CH₂—CH₂O)ₙCH₂—CH₂ group. In another preferred embodiment, n can take a value from 2 to 10, more preferably n is 2, 3, 4 or 5, and even more preferably n is 2.

When Y does not exist, the compounds of the invention have the general formula (V):

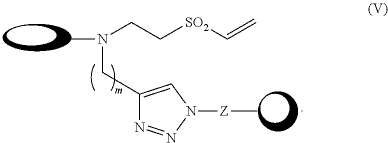

In the present invention “alkyl” refers to aliphatic chains, lineal or branched, having from 1 to 10 carbon atoms, more preferably between 1 and 5 carbon atoms, for example, methyl, ethyl, n-propyl, i-propyl, n-butyl, tert-butyl, sec-butyl, n-pentyl, etc.

The term “dialkylaryl” in the present invention refers to an aryl group substituted with two alkyl groups having from 1 to 10 carbon atoms, more preferably between 1 and 5 carbon atoms. Alkyl groups can be the same or different, preferably they are the same. The term “aryl” in the present invention refers to an aromatic carbocyclic chain, having between 6 and 12 carbon atoms, which can be of single ring or multi-ring, separated and/or condensed. Typical aryl groups contain 1 to 3 rings separated or condensed and from 6 to approximately 18 ring carbon atoms, such as phenyl, naphthyl, indenyl, phenanthryl or anthracyl radicals.

The “label molecule” in this description refers to any biorecognizable substance, colouring agent, fluorophore or any other group which can be detected through spectrophotometric, fluorometric, optical microscopy, fluorescence or confocal, antibodies and/or RMN techniques, and which easily enables the detection of another label molecules which is hard to detect and/or quantify by itself. Preferably, this label molecule is biotin or a fluorophore selected among fluorescent markers which contain or are susceptible to be derivatized for the introduction of a carboxylic acid group, a sulphonic acid group or an azide group, hereinafter the molecules being represented, with carboxylic acid group or sulphonic acid group, also according to the figures:

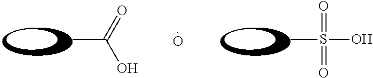

More preferably, these fluorophores are fluorescein, dansyl, rhodamine or any of their derivatives. The derivatives of label molecules can be acid halogenides or sulfonyl halogenides, and more preferably acid or sulfonyl chlorides.

In the present invention, the two label molecules which include the compounds of the invention are of a different nature.

A second aspect of the present invention refers to a method for obtaining the compounds of the invention of the general formula (IV), that is, when Y is the —SO₂R group and which comprises:

reacting:
a vinyl sulphone functionalized of general formula (II), containing at least and amine group and a terminal alkynyl group, for the binding with the label molecules,

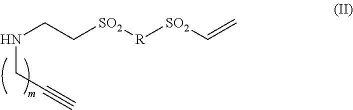

where R and m are previously defined.
with a label molecule containing a carboxylic acid group, a sulphonic acid group or any of the activated derivatives of these functions before or after reacting with an azide derived from another labelling molecule of a different nature as the previous one, according to any of the following schemes:

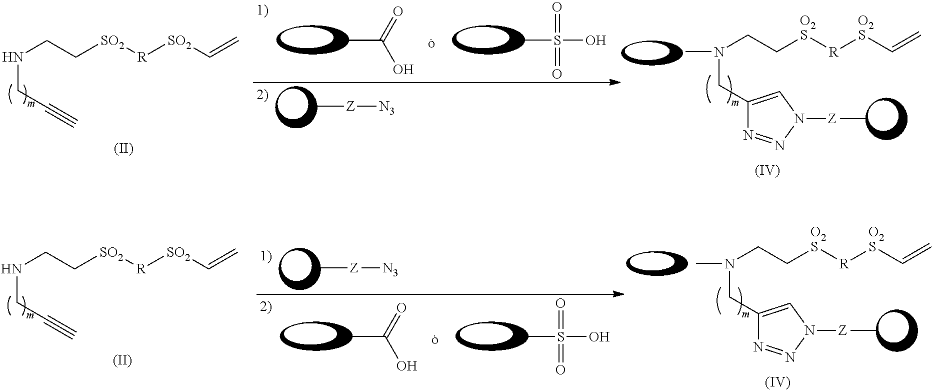

where: Z, m and R are defined above.

The sequential order of these reactions is irrelevant, the acid derived from a label molecule can react first or any of its derivatives activated with the compound of general formula (II) to form a binding of the amide or sulfonamic type and then the azide derived from another label molecule; or else the azide derived from the label molecule can react first with the compound of general formula (II) and then the acid with another label molecule or any of the activated derivatives of this function, obtaining in the same way, and in both cases, the compound of general formula (IV) which corresponds to the compound of general formula (II) of the invention when Y is the group —SO₂R.

In a preferred embodiment of the present invention, derived acid chloride or derived sulfonyl chloride from the label molecules can be used.

A third aspect of the present invention refers to a method for obtaining the compounds of the invention of general formula (V), that is, when Y does not exist, and which comprises:

reacting:
a compound derived from 2-{[ω-alkenyl alkylamine)ethyl]sulfonyl}ethanol of formula (III), containing at least a secondary amine group and a terminal alkenyl group for the binding with labelling molecules,

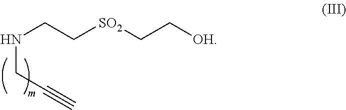

where m is defined above.
with a label molecule containing a sulfonic acid group, more preferably a derived label molecule with a sulfonyl chloride group, and later reaction with an azide derived from another labelling molecule different from the previous one:

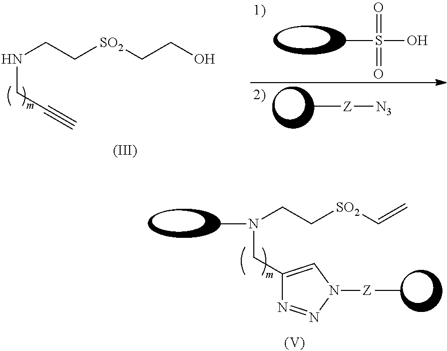

where: Z and m are defined above.

The sequential order of the reactions is the one indicated above, obtaining through this procedure compounds of the general formula (V) which correspond to the compounds of the general formula (I) of the invention when Y does not exist.

A preferred embodiment of the present invention comprises functionalized vinyl sulphones of general formula (II) where R is the group (CH₂CH₂O)ₙCH₂CH₂, n is described above; n can take a value between 2 and 10, more preferably n is 2, 3, 4 or 5 and even more preferably n is 2. These functionalized vinyl sulphones can be obtained by reaction of divinyl sulphone (DVS) with ethylene glycol (when n is 2) or polyethylene glycol derivatives (when n is higher than 2) and later reaction of one of the vinyl sulphone groups with w-alkenyl alkylamine through an addition reaction of the Michael type, as shown below:

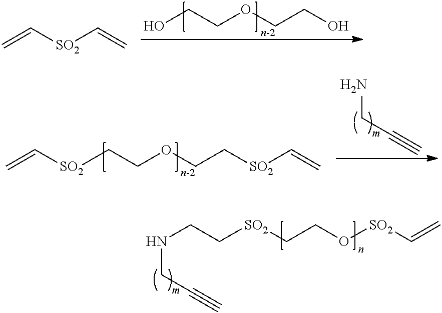

In another preferred embodiment of the present invention, the compound of formula (III) is 2-{[2-(prop-2-in-1-ilamine)ethyl]sulfonyl}ethanol which can be obtained through reaction of (2-etenilsulfonyl)ethanol and propargylamine, according to the following scheme:

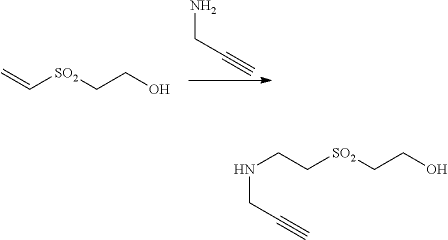

In this way, the compounds of formula (II) and (III) are trifunctional compounds with groups presenting orthogonal reactivity between each other, which is a circumstance which enables to modulate their reactivity. Thus, according to any of the methods of the present invention, vinyl sulphones of general formula (II) and (III) enable to carry out the incorporation of any label molecule containing functional groups with a complementary reactivity to the groups present in them, and which leave a vinyl sulphone group unaltered which is the one used for the later binding to the biomolecules. Particularly, and given the fact that vinyl sulphones of formulas (II) of the preferred embodiment of the present invention are carriers of the amine and alkenyl functions, there can be used, but without limiting to, derivatives of label molecules containing: a) the acid chloride function or sulfonyl chloride and b) the azide function, respectively. Alternatively, since vinyl sulphones of formulas (III) of the preferred embodiment of the invention are carriers of the amine, hydroxyl and alkenyl functions, there can be used, but without limiting to, derivatives of label molecules containing: a) the sulfonyl chloride function and b) the azide function.

In a preferred embodiment of the present invention, the label molecules used are biotin and fluorophores selected between fluorescein, dansyl or rhodamine, or any of their derivatives. An even more preferred embodiment of the present invention comprises the following acid chlorides and the sulfonyl chloride of these label molecules:

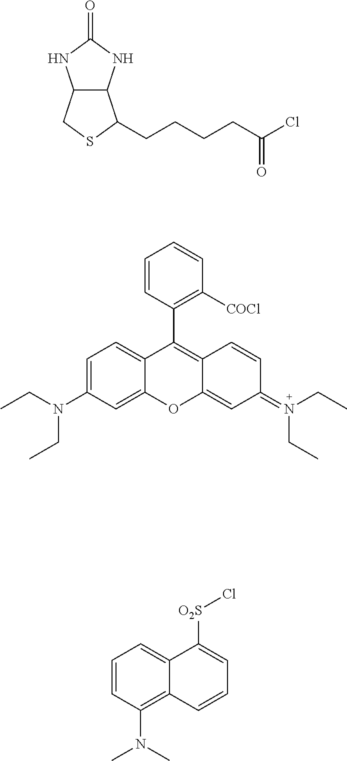

and also the azide derivatives, which are indicated below:

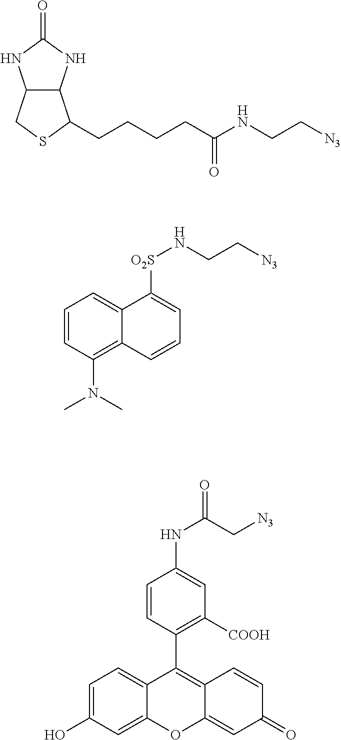

In a preferred embodiment of the present invention, obtaining the compounds of the invention, double labelling agents, is performed by reaction of the aforementioned derivatives of label molecules (acid chlorides, sulfonyl chlorides and azide derivatives) with vinyl sulphones of general formula (II) or with 2-{[ω-alkenyl alkylamine)ethyl]sulfonyl}ethanol of formula (III) through: a) N-acylation reactions with acid chlorides of the labels; or N-sulfonation reactions with sulfonyl chlorides and b) cycloaddition reactions of 1,3-dipolars with azide derived from label molecules. The sequential order of these reactions is irrelevant for the case of the first method of the invention described, although in a preferred embodiment the order is N-acylation/cycloaddition. For the case of the second method of the invention, described above, the sequential order of these reactions is N-sulfonation followed by cycloaddition.

The use of the vinyl sulphone function as derivatization of the labelling reagents to perform the covalent binding biomolecule-compound of the invention has the following advantages:
a) Stability of the labelling agents.
b) Formation of a stable covalent binding.
c) Fast reaction with high yields, not generating any type of by-product.
d) No great reagent excess required.
e) Reactions are performed in absence of catalysts through simple mixture of reagents.
f) Reactions can be performed in water without using co-solvents.
g) Reactions can be performed under low physiological conditions: aqueous medium, narrow pH range, mild temperatures.
h) Simple purification and isolation processes.
i) There exists a tolerance to other functional groups present in biomolecules other than amine and thiol groups with which vinyl sulphones react.

Therefore, another aspect of the present invention refers to the use of the compounds of general formula (I) as labelling agents for the marking or labelling of molecules, and more preferably of biomolecules. In the present invention the term “labelling agent” refers to those compounds which are able of binding to a molecule and which also allow displaying, detecting and/or quantifying by means of spectroscopy (absorption, fluorescence, NMR and others), enzymatic reactions (peroxidase, alkaline phosphatase and others) or spectrometry (mass and others) of the molecule object of the marking.

Double labelling agents containing vinyl sulphones (compounds of general formula (I)) can be bound to any biomolecule containing complementary functional groups (amino group and thiol groups) present therein naturally or artificially through a Michael type addition reaction. Besides, the compounds are compatible with the biological nature of biomolecules and the marking reaction does not require any activation strategy.

In a preferred embodiment of the present invention, the selected biomolecules are proteins. In an even more preferred embodiment of the present invention, the selected proteins are Bovine Serum Albumin (BSA), Human Serum Albumin (HAS), lysozyme, Horseradish peroxidase, artichoke peroxidase, GFP (Green Fluorescent Protein) or Concanavalin A.

In a preferred embodiment of the present invention protein labelling is carried out in a solution of these in a buffer without containing free amines such as, but without limiting to phosphate or HEPES, at moderate ionic strength, (50-200 mM) and basic pH (7.5-8.7) and the reaction with an excess of the labelling reagents of general formula (I) during an appropriate time (usually for a few hours at room temperature or all night long at 4° C.) being the reagent excess eliminated by dialysis. The labelling is performed through the following scheme:

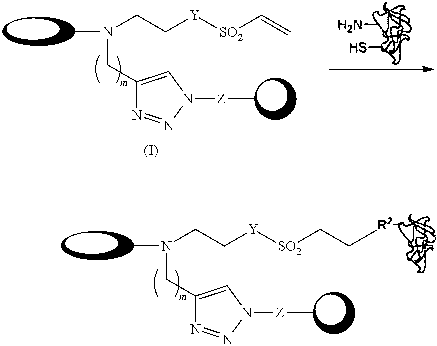

Where: Y, Z and m are previously defined;
R² can be NH or S; and
[symbol] represents a biomolecule.

Throughout the description and the claims the word “comprise” and its variants are not intended to exclude other technical features, additives, components or steps. For the subject experts, other objects, advantages and features of the invention will be inferred in part from the description and in part from the practice of the invention. The following examples and figures are provided as an illustration, and are not intended to be limitative to the present invention.

### DESCRIPTION OF THE DRAWINGS

FIG. 1 shows the fluorescence of the SDS-PAGE gel of BSA after the reaction with a double labelling agent 25 according to the reaction conditions. Rows 2, 3, 3, 4 and 5 marking at room temperature and marking reagent:protein stoichiometries of 5:1, 10:1, 25:1 and 50:1, respectively. Rows 6, 7, 8 and 9 marking at 37° C. and stoichiometries of 5:1, 10:1, 25:1 and 50:1, respectively. Row 10 BSA control (without marking).

FIG. 2 shows the emission spectrum of the double labelling agent 29 (A), of the control HAS (B) and of the marked HSA (C) after excitation at 280 nm.

FIG. 3 shows the fluorescence of the SDS-PAGE gel after the reaction with the double labelling agent 25 with the HRP (FIG. 3A) and with the artichoke peroxidase (FIG. 3B). From left to right, the marking reagent: peroxidase stoichiometries are 1:5, 1:10, 1:20, 1:30, 1:40 and 1:50.

FIG. 4 shows the fluorescence of the SDS-PAGE gel after the reaction of HRP with the double labelling agent 25 (left rows) and 27 (right rows).

### EXAMPLES

There follows an illustration of the invention by means of some assays carried out by the inventors, which prove the specificity and effectiveness of the compounds of the invention.

### Example 1

### Synthesis of Vinyl Sulphones Containing Propargyl Groups and Secondary Amines. Compounds of General Formula (II)

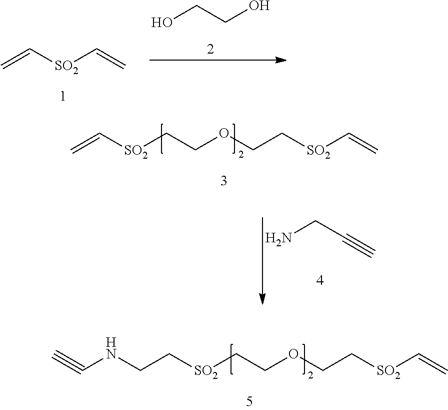

Compound 3: DVS 1(1.6 mL, 16 mmol) and t-BuOK (119 mg, 1.1 mmol) were added to a solution of ethylene glycol 2 (330 mg, 5.3 mmol) in THF (100 mL). The reaction mixture was left at room temperature (30 min.) the solvent was eliminated by vacuum evaporation. The crude obtained was purified by column chromatography (AcOEt-hexane 2:1 to 3:1) obtaining 3 as a syrup (800 mg, 51%).

Compound 5: Propargylamine 4 (51 mg, 0.93 mmol) was added to a dissolution of 3 (414 mg, 1.4 mmol) in CH₂Cl₂-isopropanol 2:1. The reaction mixture was left at room temperature (1 day). The solvent was eliminated by vacuum evaporation obtaining a crude that was purified by column chromatography (AcOEt to AcOEt-MeOH 10:1) obtaining 5 as a syrup (170 mg, 52%).

### Example 2

### Synthesis of 2-{[2-alkenyl amine)ethyl]sulfonyl}ethanol derivatives of general formula (III)

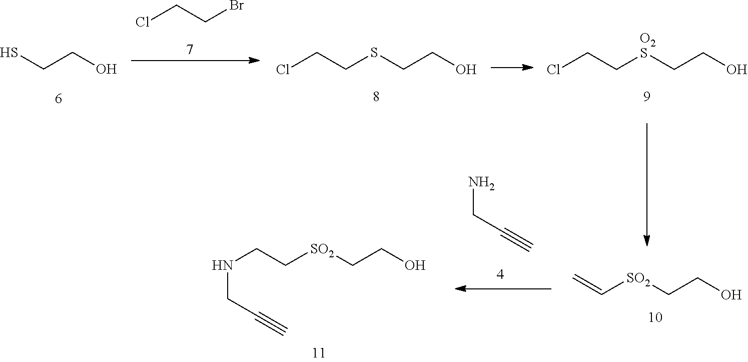

Compound 8: A mercaptoethanol dissolution 6 (300 mg, 3.84 mmol) in anhydride acetonitrile (15 mL) was deoxigenated by bubbling of Ar for 5 min. Bromochloroethane 7 (0.7 mL, 7.68 mmol) and Cs₂CO₃ (1.9 g, 5.76 mmol) were added. The reaction mixture was kept under stirring for 16 hours. After filtration of the Cs₂CO₃, the solvent was eliminated by vacuum evaporation and the resulting crude was purified by column chromatography (ether-hexane 2:1) obtaining 8 (410 mg, 76%).

Compound 9: H₂O₂ of 33% (3.4 mL) was added to the solution of 8 (237 mg, 1.68 mmol) in AcOH (8.5 mL). The reaction mixture was kept at room temperature in the dark for 1 day. After vacuum evaporation the resulting crude was purified by column chromatography (ether) obtaining 9 (182 mg, 63%).

Compound 10: Et₃N (2 mL, 14 mmol) was added to the solution of 9 (0.846 g, 4.9 mmol) in THF (10 mL). The reaction mixture was kept at room temperature (1.5 h). The solvent was eliminated by vacuum evaporation and the resulting crude was purified by column chromatography (AcOEt) obtaining 10 (540 mg, 81%).

Compound 11: Propargylamine 4 (212 mg, 3.85 mmol) was added to a dissolution of 10 (577 mg, 4.24 mmol) in THF-isopropanol 1:2 (20 mL). The reaction mixture was kept at room temperature (1 day). The solvent was eliminated by vacuum evaporation. The crude obtained was purified by column chromatography (AcOEt-MeOH 5:1) obtaining 11 as a solid (710 mg, 96%).

### Example 3

### Synthesis of Acid Chloride Derivatives

### Example 3.1

### Synthesis of Acid Chloride Derived from Biotin

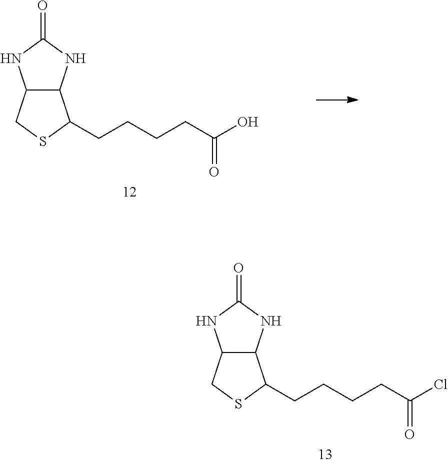

Compound 13: A solution of biotin 12 (200 mg, 0.82 mmol) in Cl₂SO (5 mL) was kept at room temperature (1 h). The excess of Cl₂SO was eliminated by vacuum evaporation successively co-evaporating with anhydride toluene. The syrup obtained corresponds to the compound 13 and is used directly without any type of purification.

### Example 3.2

### Synthesis of Acid Chloride Derived from Rhodamine B

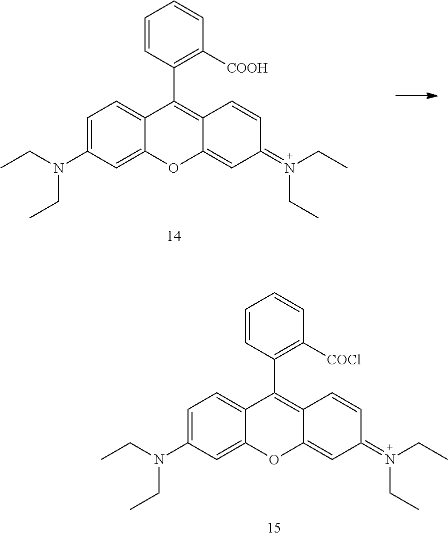

Compound 15: A solution of rhodamine B 14 (195 mg, 0.41 mmol) in POCl₃ (5 mL) and 1,2-dichloroethane (5 mL) was kept at reflux (16 h). The excess of POCl₃ and the solvent were eliminated by vacuum evaporation successively co-evaporating with anhydride toluene. The crude obtained contains rhodamine chloride 15 and is directly used without any type of purification.

### Example 4

### Synthesis of Azide Derivatives

### Example 4.1

### Synthesis of Azide Derived from Biotin 17

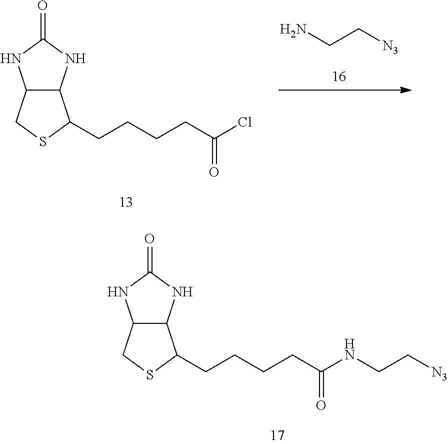

Compound 17: 2-azide ethylamine 16 (0.22 g, 2.45 mmol) and Et₃N (0.525 mL) dissolved in anhydride acetonitrile (5 mL) were added to a solution in anhydride acetonitrile (15 mL) of chloride derived from biotin 13 obtained as indicated in example 3.1 from biotin (0.3 g, 1.22 mmol). The reaction mixture was left at room temperature (15 min). The solvent was eliminated by vacuum evaporation and the resulting crude was purified by column chromatography (AcOEt-MeOH 5:1) obtaining 17 (0.28 g, 73%).

### Example 4.2

### Synthesis of Azide Derived from Dansyl 19

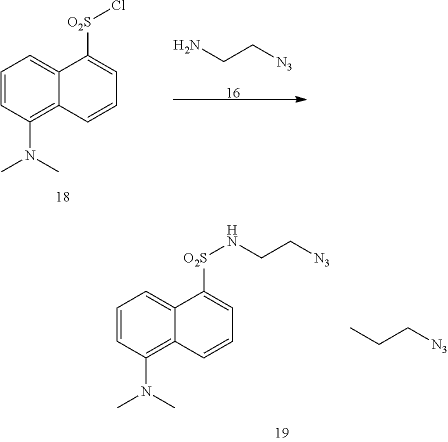

Compound 19: 2-azide ethylamine 16 (390 mg, 4.5 mmol) and Et₃N (0.5 mL, 3.5 mmol) were added to a solution of commercial dansyl chloride 18 (600 mg, 2.2 mmol) in Cl₂CH₂ (15 mL). The reaction mixture was left at room temperature (15 min). The solvent was eliminated by vacuum evaporation. The crude obtained was purified by column chromatography (ether-hexane 2:1) obtaining 19 as a solid foam (680 mg, 96%).

### Example 4.3

### Synthesis of Azide Derived from Fluorescein 22

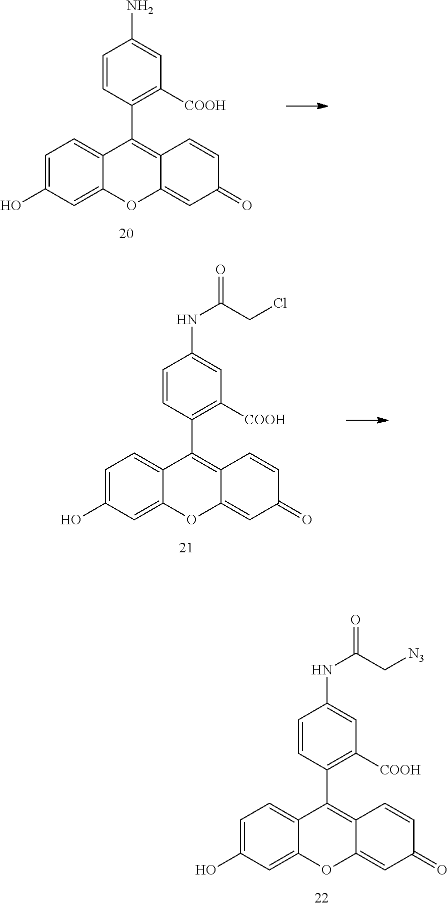

Compound 21: Choroacetic anhydride (443 mg, 2.6 mmol) was added to a solution of fluoresceinamine 20 (450 mg, 1.3 mmol) in MeOH. The reaction mixture was left at room temperature (1 day). The precipitate which appeared was filtered, washed (with MeOH and later ether) and dried obtaining 21 (440 mg, 80%).

Compound 22: Sodium azide (306 mg, 4.7 mmol) was added to a suspension of 21 (400 mg, 0.94 mmol) in MeOH (20 mL). The reaction mixture was irradiated with MW (500 W, 65° C., 10 h). The solvent was eliminated by vacuum evaporation. The crude obtained was purified by column chromatography (AcOEt, AcOEt-MeOH 1:1 to MeOH) obtaining 22 as a solid (330 mg, 82%).

### Example 5

### Synthesis of Double Labelling Agents Based on Vinyl Sulphones Containing Biotin and Fluorophores

### Example 5.1

### Synthesis of Labelling Agents 24 and 25

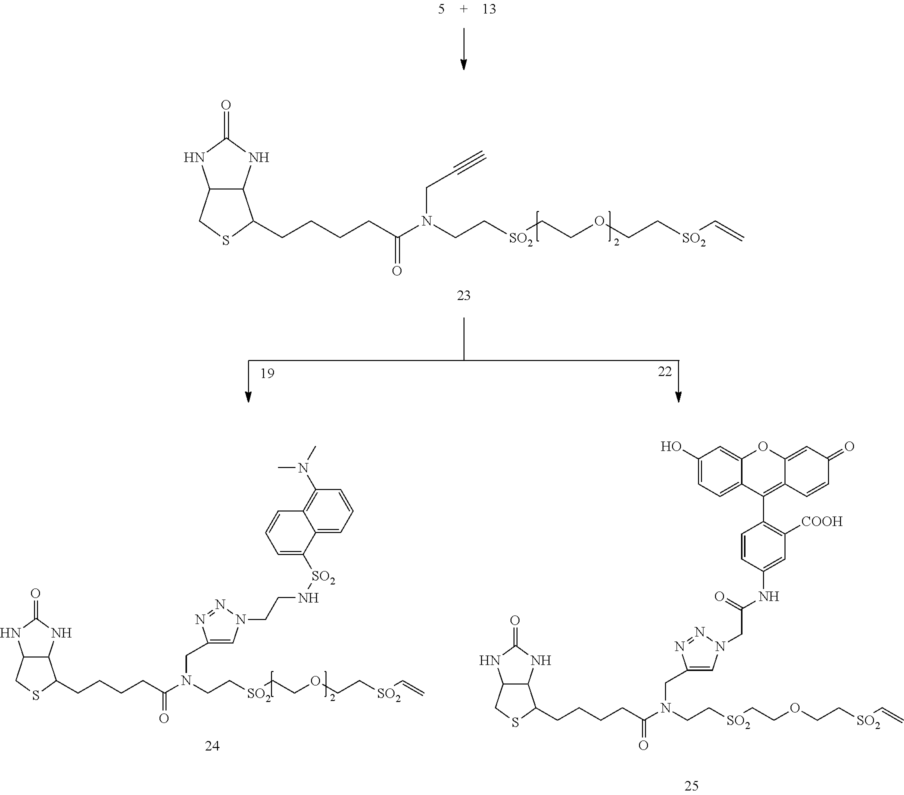

Compound 23: A solution of biotin chloride 13 in THF anhydride (15 mL), obtained from biotin (200 mg, 0.82 mmol) as indicated in example 3.1, was cooled in a water-ice bath and 5 (353 mg, 1 mmol) and Et₃N (0.230 mL, 1.6 mmol) dissolved in THF anhydride (5 mL) were added. The reaction mixture was left to reach room temperature, then the solvent was eliminated through vacuum evaporation. The crude obtained was purified by column chromatography (AcOEt-MeOH 5:1) obtaining 23 as a syrup (438 mg, 92%).

Compound 24: 19 (80 mg, 0.25 mmol), Et₃N (0.09 mL, 0.62 mmol) and CuI(C₂H₅O)₃P (8 mg, 0.022 mmol) were added to a solution of 23 (120 mg, 0.21 mmol) in MeOH (15 mL). The reaction mixture was left at room temperature (3.5 h). The solvent was eliminated through vacuum evaporation. The crude obtained was purified by column chromatography (AcOEt-MeOH 3:1) obtaining 24 as a solid (167 mg, 90%).

Compound 25: 22 (102 mg, 0.24 mmol), Et₃N (0.085 mL, 0.4 mmol) and CuI(C₂H₅O)₃P (10 mg, 0.023 mmol) were added to a solution of 23 (115 mg, 0.2 mmol) in MeOH (15 mL). The reaction mixture was left at room temperature (3.5 h). The solvent was eliminated through vacuum evaporation. The crude obtained was purified by column chromatography (AcOEt-MeOH 3:1 to MeOH) obtaining 25 as a solid (140 mg, 70%).

### Example 5.2

### Synthesis of the Labelling Agent 27

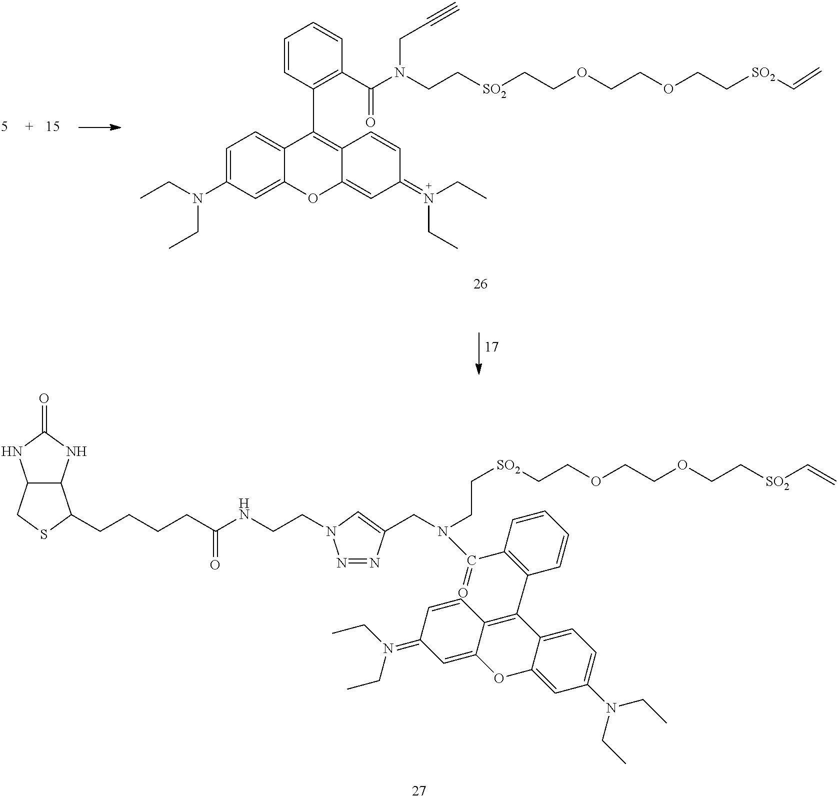

Compound 26: A solution of rhodamine B chloride 15 in THF anhydride (15 mL), obtained from rhodamine B (195 mg, 0.41 mmol) as indicated in example 3.2, was cooled in a water-ice bath and 5 (174 mg, 0.49 mmol) and Et₃N (0.174 mL, 1.22 mmol) dissolved in THF anhydride (5 mL) were added thereto. The reaction mixture was left to reach room temperature, then the solvent was eliminated through vacuum evaporation. The crude obtained was purified by column chromatography (Cl₂CH₂-MeOH 20:1) obtaining 26 as a solid (272 mg, 86%).

Compound 27: The azide derivative 17 (69 mg, 0.22 mmol), Et₃N (0.080 mL, 0.057 mmol) and CuI(C₂H₅O)₃P (10 mg, 0.029 mmol) were added to a solution of 26 (140 mg, 0.18 mmol) in MeOH (15 mL). The reaction mixture was left at room temperature (3.5 h). The solvent was eliminated through vacuum evaporation. The crude obtained was purified by column chromatography (AcOEt-MeOH 3:1 to MeOH) obtaining 27 as a solid (141 mg, 72%).

### Example 5.3

### Synthesis of the Labelling Agent 29

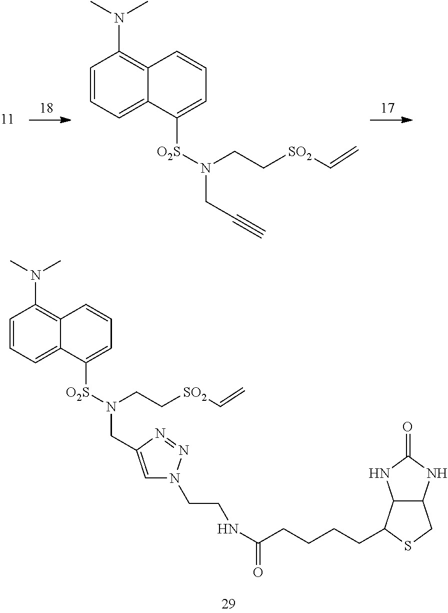

Compound 28: Dansyl chloride 18 (680 mg, 2.52 mmol) and Et₃N (0.71 mL, 5.02 mmol) were added to a solution of 11 (160 mg, 0.84 mmol) in CH₂Cl₂ anhydride (20 mL). The reaction mixture was left at room temperature (1 day). The solvent was eliminated through vacuum evaporation. The crude obtained was purified by column chromatography (AcOEt-hexane 2:3) obtaining 28 as a syrup (230 mg, 68%).

Compound 29: Compound 17 (89 mg, 0.29 mmol), Et₃N (0.080 mL, 0.57 mmol) and CuI(C₂H₅O)₃P (10 mg, 0.029 mmol) were added to a solution of 28 (128 mg, 0.31 mmol) in MeOH (15 mL). The reaction mixture was left at room temperature (2 h). The solvent was eliminated through vacuum evaporation. The crude obtained was purified by column chromatography (AcOEt-MeOH 4:1) obtaining 29 as a solid (188 mg, 92%).

### Example 6

### Double Labelling of Proteins with Biotin-Fluorophore

The labelling of proteins is performed according to the following general protocol: a solution of protein in a buffer which does not contain free amines, such as phosphate or HEPES, at moderate ionic strength (50-200 mM) and basic pH (7.5-8.7) is made to react with 5 moles of marking reagent per protein mol for enough time (normally all night at room temperature). The reagent excess is eliminated by dialysis.

### Example 6.1

### Labelling of Bovine Serum Albumin (BSA) with the Double Labelling Agent 25

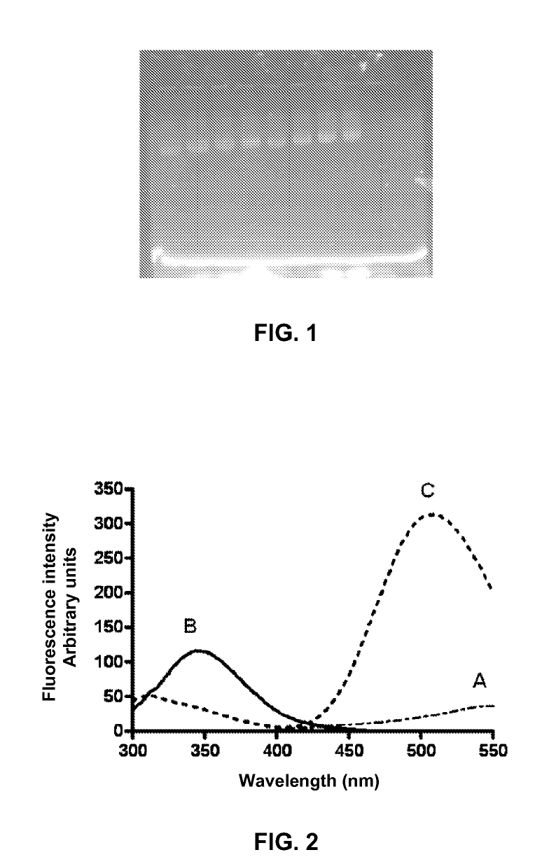

BSA has a molecular weight of 66.4 kDa and 4.8 of isoelectric point, is hydrosoluble and stable in solution so it is a good model to study optimum marking conditions. The influence of temperature (room temperature and at 37° C.) and stoichiometry (marking reagent:protein 5:1, 10:1, 25:1 and 50:1) in the marking reaction. The marking effectiveness was analysed by electrophoresis in SDS-PAGE and fluorescence was visualized with a commercial transilluminator (λ=365 nm) (FIG. 1). The result shows that both the temperature and high stoichiometry produce an increase of the molecular weight of the enzyme as a consequence of a greater number of molecules of double marking reagent joined to BSA, although for purposes of fluorescence “de visu” no significant differences are observed.

### Example 6.2

### Labelling of Human Serum Albumin (HAS) with the Double Labelling Agent 29

The serum albumin is the most abundant protein in the circulatory system, responsible for 80% of the oncotic pressure in blood and the main carrier of fatty acids, little hydrosoluble hormones or drugs which are otherwise insoluble in serum. The marking reaction was performed for 12 hours and stirring at 4° C. in carbonated buffer 0.1 M pH 9 and with a stoichiometry HSA:marking reagent 1:20. The excess of double labelling agent was blocked with ethanolamine and eliminated through dialysis against a PBS buffer.

The marking reaction was evident through FRET. The experiment was performed in a Shimadzu fluorometer RF-5301 PC with a quartz deposit of 1 mL and 1 cm lightpath. The concentration of the samples was 0.1 mg/ml (in PBS). It was excited at 280 nm which is the excitation wavelength of tryptophan present in HSA, and the emission spectrum was collected from 300 to 550 nm. The results show that the marked protein presents a typical emission spectrum of 500-550 nm as a result of the excitation of dansyl by the transmission of fluorescence energy emitted by the excited tryptophan at 280 nm of the protein itself (FIG. 2). Neither the unmarked protein nor the double labelling agent 29 present this fluorescence maximum when they are excited at 280 nm which shows the existence of FRET in the labelled protein.

### Example 6.3

### Labelling of Lysozyme with the Labelling Agent 25

The egg lysozyme has a molecular weight of 14.3 kDa, an isoelectric point of around 11 and is water soluble. Its isoelectric point and low molecular weight make a good model to complement the studies performed with BSA. The influence of temperature (room temperature/37° C.) and stoichiometry (marking reagent:protein 5:1, 10:1, 25:1 and 50:1) in the marking reaction was studied. The effectiveness of the marking was analysed through electrophoresis in SDS-PAGE and the fluorescence was visualized with a commercial transilluminator (λ=365 nm), verifying that both temperature and high stoichiometry produce a decrease in solubility due to a greater number of molecules of double marking reagent bound to a lysozyme. The best results were obtained when the marking reaction was performed at room temperature and stoichiometry was 5:1.

### Example 6.4

### Horseradish and Artichoke Peroxidases with the Double Labelling Agents 24, 25, 27 and 29

Peroxidases are enzymes which catalyse the reduction of hydrogen peroxidase with the help of a substrate which loses two hydrogen atoms. They are widely used in clinical biochemistry. Also, glycoproteins are a good model to evaluate the capacity of the marking reagent to react with protected proteins through a “cover” of carbohydrates. Horseradish and artichoke peroxidases were selected because the former is the reference peroxidase in biotechnological applications and the latter has great resistance to the action of proteases, probably as a result of a greater density of carbohydrates.

Marking experiments of horseradish peroxidase were performed with reagents 24 and 29 in buffer HEPES 100 mM pH 8.5 at two temperatures (room temperature and 37° C.) and three stoichiometry labelling agent:protein (5:1, 10:1 and 50:1). The effectiveness of the marking was analysed through electrophoresis in SDS-PAGE and fluorescence was visualized with a commercial transilluminator (λ=365 nm). The results show the importance of the marking temperature, as none of the two peroxidases was marked at room temperature regardless of the stoichiometry, but they did at 37° C.

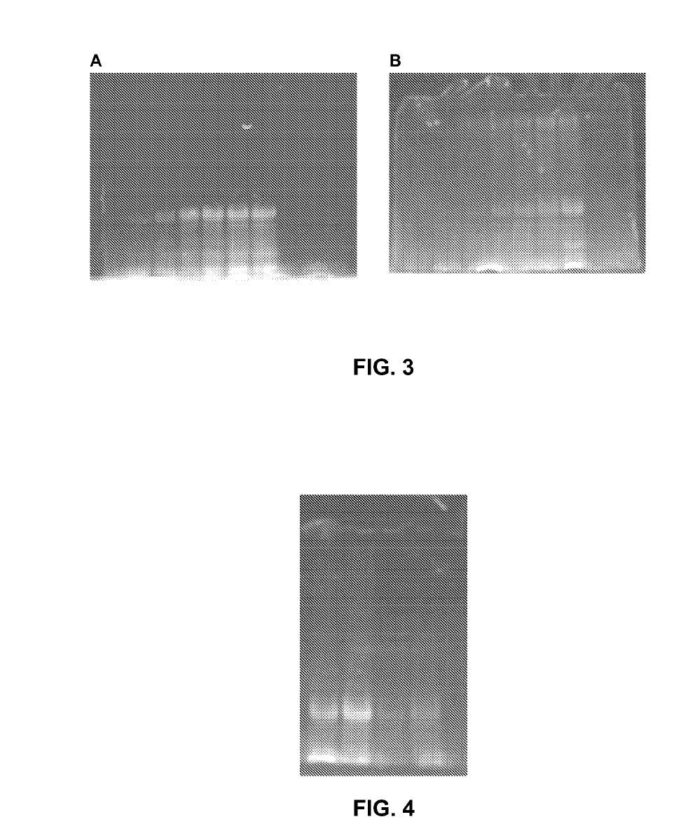

The marking of 2 mg of both peroxidases at 37° C. (1 day) in buffer HEPES 100 mM, pH 8.5, with the reagent 25 and stoichiometry protein: marking reagent 1:5, 1:10, 1:20, 1:30, 1:40 and 1:50) was studied. The samples were analysed through electrophoresis in SDS-PAGE and fluorescence was visualized with a commercial transilluminator (λ=365 nm). The marking reagent reacts, although depending on the stoichiometry and on the peroxidase: high stoichiometry originates greater fluorescence and HRP (FIG. 3A) is marked better than artichoke peroxidase (FIG. 3B). HRP, 2 mg/mL in HEPES 100 mM, pH 8.5, was made react with marking reagents 25 and 27 using two different stoichiometry (protein:marking reagent 1:25 and 1:50) and 37° C. (1 day). The samples are dialysed against phosphate buffer 50 mM, pH 7.5, NaCl-100 mM to eliminate the excess of marking reagent and analysed through electrophoresis in SDS-PAGE and the fluorescence was visualized with a commercial transilluminator (λ=365 nm). Both marking reagents reacted (FIG. 4), and just like in the preceding cases, the greater the stoichiometry, the greater the fluorescence. The effect of the binding of double labelling reagents on the activity and capacity to interact with avidin was analysed. The specific activity of the marked peroxidases is of around 65% of the unmarked ones, said value being within the range described by the provider (SIGMA) for solutions of peroxidase in buffer pH 8 after 10 days, which is the time elapsed from the beginning of the marking until the study of the activity. The interaction with avidin was shown incubating immobilized avidin on ELISA plate wells with the marked enzymes, verifying, after washing, the presence of peroxidase activity as a result of the interaction of avidin with peroxidases through the biotin of the marking, showing the functionality of biotin of the double marking reagents.

1. Compound of the general formula (I): 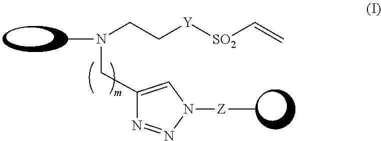
wherein:
Y is the —SO₂R— group or does not exist; where R is a radical, substituted or non-substituted, selected from the group comprising an alkyl (C₁-C₁₀), a dialkylaryl ((C₁-C₁₀)Ar(C₁-C₁₀)) or a group (CH₂—CH₂O)ₙCH₂—CH₂; where n takes values from 2 to 20;
Z is a radical, substituted or non-substituted, selected from the group comprising an alkyl (C₁-C₁₀), a dialkyl aryl ((C₁-C₁₀)Ar(C₁-C₁₀)) or a group (CH₂—CH₂O)ₙCH₂—CH₂; where n takes values from 2 to 20,
m takes values from 1 to 20; and 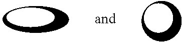
represent, independently, a label molecule.

2. Compound according to claim 1, where the label molecules are selected between biotin, fluorophore or any of their derivatives.

3. Compound according to claim 2, where fluorophore is selected between dansyl, fluorescein, rhodamine or any of their derivatives.

4. Compound according to claim 1, where Z is an alkyl (C₁-C₅).

5. Compound according to claim 4, where Z is an ethyl group.

6. Compound according to claim 4, where Z is a methyl group.

7. Compound according to claim 1, where m is 1.

8. Compound according to claim 1, where Y is the —SO₂R— group.

9. Compound according to claim 1, where R is the (CH₂—CH₂O)ₙCH₂—CH₂ group, n is defined in claim 1.

10. Compound according to claim 9, where n is 2.

11. Compound according to claim 1, where Y does not exist.

12. Compound according to claim 1, of formula:

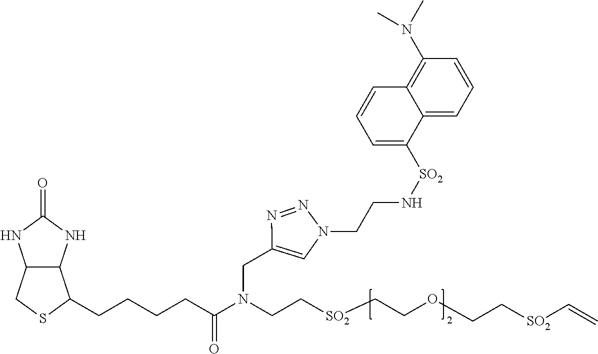

13. Compound according to claim 1, of formula:

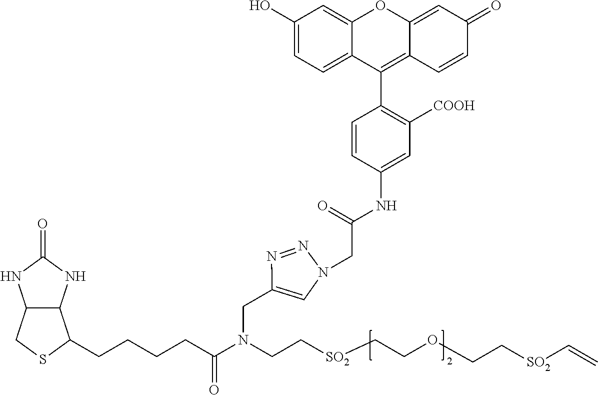

14. Compound according to claim 1, of formula:

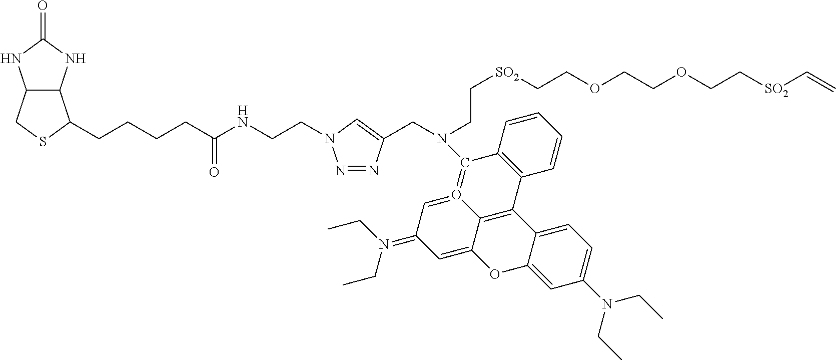

15. Compound according to claim 1, of formula:

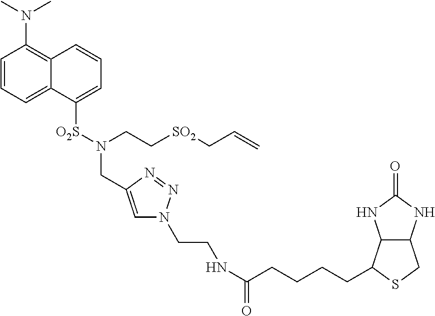

16. Method of obtaining the compounds of general formula (I) when Y is the —SO₂R— group comprising the reaction of the compound of general formula (I) 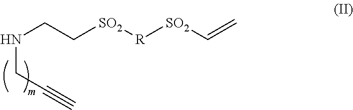
where R and m are defined in claim 1
with a label molecule or any of its derivatives, containing an acid or sulfonyl group, before or after reacting with another label molecule or any of its derivatives other than the preceding one containing an azide group.

17. Method according to claim 16, where the label molecule derivatives containing an acid or sulfonyl group are acid chlorides or sulfonyl chlorides.

18. Method according to claim 16, where R is the (CH₂—CH₂O)ₙCH₂—CH₂ group, n is defined in claim 1.

19. Method according to claim 18, where n is 2.

20. Method according to claim 16, where m is 1.

21. Method of obtaining the compounds of general formula (I) when Y does not exist, comprising:
a. reaction of the compound of general formula (III) with a label molecule or any of its derivatives, containing a sulfonyl group: 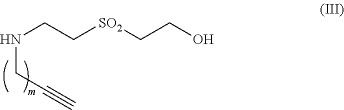
where m is defined above; and
b. reaction of the compound obtained in step (a) with another label molecule or its derivatives, other than the previous one containing an azide group.

22. Method according to claim 21, where the derivatives of the label molecules containing a sulfonyl group are sulfonyl chloride.

23. Method according to claim 21, where m is 1.

24. Use of a compound of general formula (I) according to claim 1 as a labelling agent.

25. Labelling agent comprising a compound according to claim 1.

26. Use of a labelling agent according to claim 25 for the marking of biomolecules.

27. Use of an agent according to claim 26, where the biomolecules are proteins.
